(19) United States
(12) Patent Application Publication
(10) Pub. No.: US 2025/0288302 A1
(43) Pub. Date: Sep. 18, 2025

(54) EXPANDABLE EMBOLIC IMPLANTS WITH FOLDING STRUTS

(71) Applicant: Polyembo, LLC, Park City, UT (US)

(72) Inventors: Daniel J. Ashurst, Mead, CO (US); Shawn P. Fojtik, Park City, UT (US); Joseph R. Steele, Houston, TX (US)

(21) Appl. No.: 19/080,772

(22) Filed: Mar. 14, 2025

Related U.S. Application Data
(60) Provisional application No. 63/565,501, filed on Mar. 14, 2024.
(60) Provisional application No. 63/569,145, filed on Mar. 23, 2024.
(60) Provisional application No. 63/698,580, filed on Sep. 25, 2024.

(51) Int. Cl. A61B 17/12; A61B 17/00

(52) U.S. Cl. CPC A61B 17/12145; A61B 2017/00526

(57) ABSTRACT

An embolic implant formed from a tube includes one or more expandable sections with cuts, or slits, that define struts. The slits of each expandable section are arranged to define struts that twist and fold as the expandable section of the embolic implant expands. The slits may be arranged in rows that extend helically around the tube, along a portion of its length. The slits of one row may be longitudinally offset from the slits of each circumferentially adjacent row. Methods for making embolic implants are also disclosed.

### CROSS-REFERENCE TO RELATED APPLICATIONS

Claims for priority are made to the following applications pursuant to 35 U.S.C. § 119(e): U.S. Provisional Patent Application No. 63/565,501, filed on Mar. 14, 2024 and titled EXPANDABLE OCCLUSIVE DEVICES (“the '501 Provisional Application”); U.S. Provisional Patent Application No. 63/569,145, filed on Mar. 23, 2024 and titled EXPANDABLE EMBOLIC DEVICES (“the '145 Provisional Application”); and U.S. Provisional Patent Application No. 63/698,580, filed on Sep. 25, 2024 and titled EXPANDABLE EMBOLIC IMPLANTS WITH FOLDING STRUTS (“the '580 Provisional Application”). The entire disclosures of the '501 Provisional Application, the '145 Provisional Application, and the '580 Provisional Application are hereby incorporated herein.

### TECHNICAL FIELD

This disclosure relates generally to embolic implants and, more specifically, to embolic implants that are expandable within a vasculature of a subject to permanently occupy a portion of the vasculature. More specifically, this disclosure relates to embolic implants with slits that define struts that fold upon expansion of the expandable section. Even more specifically, an embolic implant of this disclosure may include an expandable section with a flower configuration that includes differently sized loops. This disclosure also relates to methods of manufacturing embolic implants and to methods of using embolic implants.

### RELATED ART

Occlusive devices, including coils and plugs, are used to occupy blood vessels and voids within a subject's body. Occlusive devices may therapeutically and/or diagnostically slow or stop the flow of blood though blood vessels or occlude other voids within a subject's body. Occlusive devices may be used for a variety of purposes, including treating arteriovenous malformations, controlling bleeds, closing perforations, blocking aneurysms, devascularization and isolated treatment of tumors, and other conditions.

Occlusive devices, such as coils, are typically self-expanding devices designed to be constrained in a loading device, pushed through a tubular delivery device to a target location, where the occlusive device self-expands to occlude the target location. Existing occlusive devices include coils, which may be manufactured from metal or polymer. The coils may occlude blood flow on their own, or they may be supplemented with other occlusive features.

Occlusive devices may be manufactured to form any of a number of different three-dimensional shapes, or tertiary shapes, when deployed, such as a coiled tube shape or a variety of other shapes, such as an asymmetrical helical shape (e.g., a funnel shape, etc.), a spherical shape, or the like. The tertiary shape of an occlusive device may enable it to serve as a primary occlusion or enable it to be used with other occlusive devices to occlude a vessel. For example, a first occlusive device may be anchored in place and other occlusive devices be packed behind the first occlusive device.

While existing occlusive devices are useful, the extent to which they occlude blood flow is limited by the extent to which their basic structures enable them to pack together. Occlusion of a blood vessel with conventional occlusive devices typically requires the placement of five or more of the conventional occlusive devices in proximity to each other within the blood vessel.

### SUMMARY

An embolic implant of this disclosure includes a tube with at least one expandable section. The embolic implant may have an unexpanded arrangement and an expanded arrangement. The unexpanded arrangement may also be referred to as a collapsed state of the embolic implant. The expanded arrangement may also be referred to as an occluding state of the embolic implant. In the unexpanded arrangement, the embolic implant may have a tubular configuration, which may have substantially the same dimensions (e.g., outer diameter (OD), inner diameter (ID), length, etc.) as the corresponding dimensions of the tube from which the embolic implant is formed. When the embolic implant is expanded to the expanded arrangement, the length of the embolic implant may shorten and its outer diameter may increase. The expanded embolic implant provides a frame, which may facilitate occlusion (e.g., clotting, etc.) within the body of a subject at the location where the embolic implant resides. The frame may also carry optional components of the embolic implant, which may enhance the physical barrier provided by the embolic implant and/or further promote occlusion by the body of the subject. In some embodiments, when the embolic implant expands to its expanded arrangement, it may assume a configuration that resembles a flower, which is referred to herein as a “flower configuration.” Such a configuration may include loops of two or more sizes, including inner loops that extend to intermediate locations between the longitudinal axis of the expanded embolic implant and outer loops that extend to an outer extent of the extended embolic implant.

The tube of the embolic implant may comprise metal (e.g., a hypotube, etc.) or a polymer. The material from which the tube is formed may facilitate self-expansion of the expandable section of the embolic implant. In embodiments where the tube comprises a hypotube, the metal may comprise a shape memory alloy, such as nitinol (i.e., nickel-titanium alloy), which may be shape set to assume a certain shape (e.g., the expanded arrangement of the embolic implant, such as the flower configuration, etc.) when exposed to a certain condition (e.g., body temperature, etc.). An embolic implant formed from a hypotube that comprises a shape memory alloy may self-expand. In other embodiments (e.g., embodiments where the tube is a hypotube that comprises stainless steel, the tube is formed from a polymer, etc.), the embolic implant may be pre-shaped such that it expands when it is removed from a constraint (e.g., that of a delivery device, etc.) or the embolic implant may be mechanically expanded once it is delivered to a target location.

Each expandable section of the embolic implant includes an arrangement of cuts, or slits, through a wall of the tube. The slits define struts that extend along at least a portion of the length of the tube. Each strut may have a somewhat rectangular shape, which may enable the strut or portions thereof to occlude a void or passage. The slits may be oriented parallel to one another and in rows in which slits are arranged end-to-end. The rows of slits and the slits of each row may extend helically around the tube. The slits of a row of slits may be offset from the slits of each circumferentially adjacent row of slits. The offsets may be arranged as a so-called “running bond pattern” of slits, in which each slit extends along about half of a length of each circumferentially adjacent slit.

As an expandable section expands to its expanded arrangement, the struts may rotate about their longitudinal axes (i.e., twist) and fold into loops in which different locations of a surface of the strut along a length of the strut may face in different directions. Upon expanding in an unconstrained environment (e.g., outside of a vessel, etc.), the struts may be helically constrained and axially compressed to define loops that fold substantially centrally upon themselves. Together, the helical constraint and axial compression causes the struts to form a flat, hollow, disk-like superstructure. Thus, the expandable section may expand to define a disk. When expanded in a constrained environment (e.g., inside of a vessel of other void or passage, etc.), the disk may not be as large and flat. It may instead resemble a mass of collapsed mesh and, thus, may be referred to as a “mesh disk.”

In some embodiments, at an apex of each loop (e.g., a midpoint of each loop, etc.), an edge of the loop may face outwardly, placing the apex of the loop in a somewhat radial orientation. With the apex of the loop in the somewhat radial orientation, the edge of the apex of the loop may engage an inner surface of a wall (e.g., a wall of a vessel, etc.) against which the apex of the loop is positioned, which may optionally anchor the embolic implant in place at the target location (e.g., within a vessel, etc.). Locations of each loop midway between its base (e.g., an unexpanded segment of the tube, etc.) and its apex may be oriented transverse to a longitudinal axis of the tube. When the longitudinal axis of the tube of the embolic implant is oriented along a direction in which blood flows through the target location, these surfaces of the strut may be oriented transverse to the direction in which blood flows, which may enable these surfaces of the strut to disrupt the flow of blood through the target location.

In embodiments where the shape of the expanded arrangement of each expandable section of the embolic implant is preset, the set shape of an expandable section may cause the expandable section to twist about a longitudinal axis of the tube from which the embolic implant is formed and, thus, twist about a longitudinal axis of the embolic implant. Thus, as the embolic implant expands from its unexpanded arrangement to its expanded arrangement, at least a portion of the embolic implant may twist (e.g., up to about 30°, with the helical rotation of the slits, etc.) around the longitudinal axis of the embolic implant. Rotation of the embolic implant as it expands may enable struts to twist and fold in a desired manner.

In some embodiments, the embolic implant may include a plurality of expandable sections. More specifically, the plurality of expandable sections may be defined at different locations along a length of the tube.

In a specific embodiment, an embolic implant may include a hypotube and at least one expandable section along a length of the hypotube. The hypotube may include a wall and have a length with distal end and a proximal end. The wall of the hypotube may define a lumen, which extends through the length of the hypotube. The expandable section may be defined by a plurality of slits cut into the wall, extending along the length of the hypotube, and oriented parallel to each other and helically around the hypotube.

The plurality of slits of such an embolic implant may include first slits and second slits. The first slits may define first rows of slits, with each first row of the first rows of slits including at least one first slit. The second slits may define second rows of slits, with each second row of the second rows of slits including at least one second slit positioned circumferentially adjacent to a portion of at least one first slit of each circumferentially adjacent first row. The first rows of slits and second rows of slits may alternate with each other around a circumference of the hypotube to define a plurality of struts. More specifically, each first row may include a proximal first slit and a distal first slit that are oriented end-to-end, while each second row may include a second slit positioned circumferentially adjacent to a distal portion of the proximal first slit and adjacent to the distal first slit of each circumferentially adjacent first row. Additionally, each second row may lack a slit circumferentially adjacent to a proximal portion of the proximal first slit of each circumferentially adjacent first row. The lack of a slit between the proximal portions of the proximal first slits of the first rows may result in proximal struts that are wider than the struts that are defined between the first rows and second rows. The wider proximal struts may stiffen a proximal side of the expandable section relative to intermediate and/or distal sections of the expandable section, which may facilitate advancement of the embolic implant to a target location.

Optionally, such an embolic implant may include another expandable section. The expandable section may also be referred to as a first expandable section or as a proximal expandable section, while the other expandable section may also be referred to as a second expandable section or as a distal expandable section. The proximal expandable section and distal expandable section may be defined by different sections of the length of the hypotube. The distal expandable section may be defined by another plurality of slits cut into the wall of the hypotube. The other plurality of slits may include first slits and second slits. The first slits may define first rows of slits, with each first row of slits including at least one first slit. The second slits may define second rows of slits, with each second row of slits including at least one second slit positioned circumferentially adjacent to a portion of a first slit of each circumferentially adjacent first row of slits. The first rows and second rows of the distal expandable section may be continuous with first rows and second rows of proximal expandable section. More specifically, each first row of slits of the distal expandable section may include a proximal first slit and a distal first slit, with the proximal first slit being a distal portion (e.g., about half, etc.) of a distal first slit of the proximal expandable section, while each second row of slits may include a proximal second slit and a distal second slit, with the proximal second slit being positioned circumferentially adjacent to the proximal first slit and a distal portion of the distal first slit of the distal expandable section and the distal second slit being positioned adjacent to a distal portion of the distal first slit of the distal expandable section. The proximal second slit may have a length that is substantially the same as the length of the distal first slit. In addition, a length of the distal second slit may be substantially the same as the length of the distal portion of the second slit that comprises the proximal second slit of the distal expandable section.

As a further option, such an embolic implant may include an additional expandable section, which may also be referred to as a third expandable section. The additional, or third, expandable section may be isolated from the first expandable section and the second expandable section. The additional expandable section may include third rows of and fourth rows of slits. The slits may be arranged similarly to the slits of the first expandable section and the second expandable section to define struts that will twist and fold upon expansion of the third expandable section. Embodiments of embolic implants with further expandable sections (e.g., four, five, six, etc.) are also within the scope of this disclosure.

Such embolic implants may have an expanded arrangement in which first struts of the plurality of struts of the at least one expandable section fold into inner loops that extend to a first radius and second struts of the plurality of struts of the at least one expandable section that fold into outer loops that extend to a second radius that exceeds the first radius.

Embodiments of embolic implants of this disclosure may include additional features that facilitate embolization within a body of a subject. As an example, an embolic implant may include a filler. While the embolic implant is in its unexpanded arrangement, the filler may be confined or substantially confined within a lumen of the tube form which the embolic implant is formed. As the embolic implant expands, the filler may substantially remain within an interior of the embolic implant, while being exposed as struts of the embolic implant twist and fold and slits in the tube open up. The filler may supplement the ability of each expandable section of the embolic implant to physically occlude the flow of fluid (e.g., an aqueous fluid, such as blood, etc.). The filler may also optionally absorb the fluid. In some embodiments, the filler may comprise fibers that extend along the entire length of the tube while the embolic implant is in its unexpanded state. Proximal ends of the fibers may be constrained within the proximal end of the tube from which the embolic implant is formed; distal ends of the fibers may be constrained within the distal end of the tube from which the embolic implant is formed. As the embolic implant expands and its length shortens, the fibers may bunch up and optionally twist within each expandable section of the embolic implant. The fibers may be distributed substantially evenly through the interior of each expanded expandable section of the embolic implant.

A method of manufacturing an embolic implant may include cutting a plurality of rows of slits into a tube (e.g., a hypotube formed from a shape memory alloy, a hypotube formed from stainless steel, a tube formed from a polymer, etc.) to define an expandable section from the tube. Optionally, cutting may comprise laser cutting the tube. Each slit of the plurality of rows of slits may extend substantially along a longitudinal axis of the tube. Optionally, the plurality of rows of slits may extend helically around the tube (e.g., at an angle of about 30° or less to a longitudinal axis of the tube, at an angle of about 20° or less to the longitudinal axis of the tube, at an angle of about 10° or less to the longitudinal axis of the tube, etc.) (e.g., the slits may be oriented in a clockwise helix around the tube, etc.). The slits may be cut to define alternating first rows of slits and second rows of slits in the tube. Each first row of slits may include a series of first slits arranged end-to-end. Each second row of slits may include a series of second slits arranged end-to-end. Each second slit may be longitudinally offset from circumferentially adjacent first slits of each adjacent first row of slits. The first rows of slits and second rows of slits define struts of the expandable section, with an arrangement of the first rows of slits and the second rows of slits enabling the expandable section to expand to an expanded arrangement. Without limitation, the expanded arrangement may comprise a flower configuration, in which first struts of the struts of the expandable section fold into inner loops that extend to a first radius and second struts of the struts of the expandable section fold into outer loops that extend to a second radius that exceeds the first radius.

The method may also include expanding the expandable section to its expanded arrangement and setting a shape of the expanded arrangement. In embodiments where the tube comprises a shape memory alloy, the shape may be set thermally, which may enable the shape memory alloy to assume its expanded arrangement when the shape memory alloy is heated to a shape memory temperature (e.g., body temperature, etc.) and the expandable section is removed from any physical constraint (e.g., a delivery device, etc.). In some embodiments where the tube comprises another material (e.g. stainless steel, a polymer, etc.), the shape of the expanded arrangement may be set mechanically to make the expanded arrangement the relaxed state of the expandable section; the expandable section may then be constrained (e.g., in another tube, etc.) until it is delivered to a target site within a body of a subject. In other embodiments where the tube comprises the other material (e.g., stainless steel, a polymer, etc.), the expandable section may be substantially tubular in its relaxed state, then mechanically forced into and secured in its expanded arrangement once it is delivered to the target site within the subject's body. In each embodiment, placement of the expandable section into its expanded arrangement may include forcing opposite ends of the expandable section and, optionally, rotating one end of the expandable section about the longitudinal axis of the tube and relative to the other end of the expandable section or rotating one end of the tube about the longitudinal axis relative to the other end of the tube. The end of the tube may be rotated in the same direction as the helices that are defined by the slits and struts formed from the tube rotate. Such rotation may be up to about 30°.

In some embodiments, fibers may be introduced into the tube from which the embolic implant is defined and constrained within ends of the embolic implant.

One or more embolic implants of this disclosure may be introduced to a target location within a body of a subject with a delivery device and through a catheter. Once the embolic implant is properly positioned and the expandable section(s) of the embolic implant has expanded, the embolic implant may be released by the delivery device, and the delivery device and the catheter may be removed from the subject's body.

More specifically, introduction of the embolic implant may be accomplished by introducing a delivery device (e.g., a catheter, a sheath, a cannula, a needle, etc.) into the body of a subject and advancing a distal end of the delivery device to a target location (e.g., to the target location, adjacent to the target location, proximally adjacent to the target location, etc.) within the body of the subject. Introduction and advancement of the delivery device may occur prior to placing an embolic implant and a control wire within the delivery device or while the embolic implant and the control wire reside within a lumen of the delivery device. With the distal end of the delivery device at or adjacent to the target location, the control wire may be advanced distally to push the embolic implant through the distal end of the delivery device.

As the embolic implant exits the distal end of the delivery device, it is introduced into the target location. As the embolic implant is introduced into the target location or shortly after the embolic implant is introduced into the target location, the embolic implant expands. More specifically, an outer diameter of a tube of the embolic implant expands and a length of the tube of the embolic implant contracts, placing the embolic implant in an expanded state. Such expansion may occur as the embolic implant exits the distal end of the delivery device, upon releasing a constraining force placed by the delivery device on the embolic implant and/or upon exposing the embolic implant to an expanding condition present at the target location (e.g., body temperature, etc.). As the embolic implant expands, it may also assume a final shape, which is at least partially defined by the cuts of in the tube of the embolic implant and may also be at least partially defined by the boundaries of the space in which the embolic implant expands. In some embodiments, including those where the embolic implant includes a plurality of closely positioned expandable sections, mesh disks of the embolic implant may pack upon each other, or mesh together; thus, the embolic implant may pack upon itself in its final shape.

The method may optionally include repositioning the embolic implant by collapsing the embolic implant, moving it, or repositioning it, and then reexpanding it.

Once the embolic implant has expanded at its target location, the control wire may be disconnected from the embolic implant. As an example, the embolic implant may be disconnected from the control wire by pulling the control wire proximally to pull the embolic implant against the proximal end of the delivery device with sufficient force to allow the embolic implant to break away from or otherwise uncouple from a distal portion of the control wire. As another example, expansion of the embolic implant may at least partially facilitate disconnection of the embolic implant from the distal portion of the control wire. Once the distal portion of the control wire disengages the embolic implant (e.g., an interior of the embolic implant, etc.), the control wire may be withdrawn proximally through the delivery device. Optionally, the delivery device may be used to introduce one or more embolic implants to the target location. Once use of the delivery device is no longer required, the delivery device may be withdrawn from the body of the subject.

Other aspects of the disclosed subject matter, as well as features and advantages of various aspects of the disclosed subject matter, will become apparent to those of ordinary skill in the art through consideration of the preceding disclosure, the ensuing description, the accompanying drawings, and the appended claims.

### BRIEF DESCRIPTION OF THE DRAWINGS

In the drawings:

FIG. 1 is a perspective view of an embodiment of an implant with a plurality of adjacent expandable sections in expanded arrangements;

FIG. 2 is a side view of the embodiment of embolic implant shown in FIG. 1 with its expandable sections in unexpanded arrangements;

FIG. 3 is a flat drawing showing a pattern of cuts, or slits in a tube to define the expandable sections of the embodiment of embolic implant shown in FIG. 2;

FIG. 4 is a side view of the embodiment of embolic implant of FIG. 2 with its expandable sections in expanded arrangements;

FIG. 5 is an end view of the embodiment of embolic implant of FIG. 2 with its expandable sections in expanded arrangements;

FIG. 6 is a perspective view of another embodiment of an implant with a plurality of adjacent expandable sections in expanded arrangements;

FIG. 7 is a side view of the embodiment of embolic implant shown in FIG. 6 with its expandable sections in unexpanded arrangements;

FIG. 8 is a flat drawing showing a pattern of cuts, or slits in a tube to define the expandable sections of the embodiment of embolic implant shown in FIG. 7;

FIG. 9 is a side view of the embodiment of embolic implant of FIG. 7 with its expandable sections in expanded arrangements;

FIG. 10 is an end view of the embodiment of embolic implant of FIG. 7 with its expandable sections in expanded arrangements;

FIG. 11 illustrates is a side view of embodiment of an embolic implant that includes fibers extending along a length of the embolic implant;

FIG. 12 is a cross-sectional representation of the embodiment of embolic implant shown in FIG. 11;

FIG. 13 is an end view of the embodiment of embolic implant shown in FIGS. 11 and 12 in its expanded arrangement outside of a confined space; and

FIG. 14 is an end view of the embodiment of embolic implant shown in FIGS. 11 and 12 in expanded arrangement within a confined space, such as a blood vessel.

### DETAILED DESCRIPTION

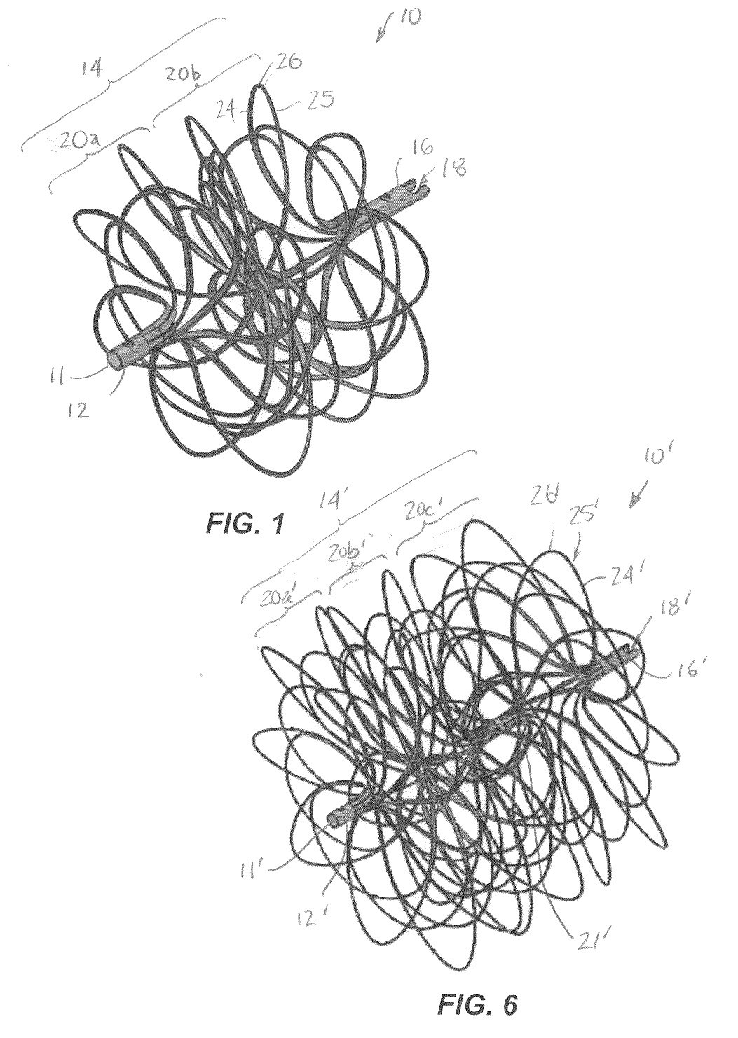

An embodiment of an embolic implant 10 is illustrated by FIG. 1. The embolic implant 10 includes a distal end 12, an intermediate section 14, and a proximal end 16. The intermediate section 14 may be directly adjacent to the distal end 12. The proximal end 16 is on an opposite side of the intermediate section 14 from the distal end 12. The proximal end 16 may be directly adjacent to the intermediate section 14.

The intermediate section 14 of the embolic implant 10 includes at least one expandable section 20. In the embodiment of embolic implant 10 illustrated by FIG. 1, the intermediate section 14 includes a pair of expandable sections 20a and 20b that, as illustrated, are expanded and, thus, in expanded arrangements.

The proximal end 16 of the embolic implant 10 may include a release 18, which enables the embolic implant 10 to be coupled to a delivery device (not shown) that delivers the embolic implant to a target location (e.g., a location within a body of a subject, etc.). The release 18 may have a configuration that enables the embolic implant 10 to be released by the delivery device once the embolic implant 10 has been advanced to and, optionally, positioned within the target location. In some embodiments, a configuration of the release 18 may also enable the delivery device to reengage the embolic implant 10 to facilitate its repositioning within the target location, movement within the target location or to another location, or removal (e.g., from the body of the subject, etc.).

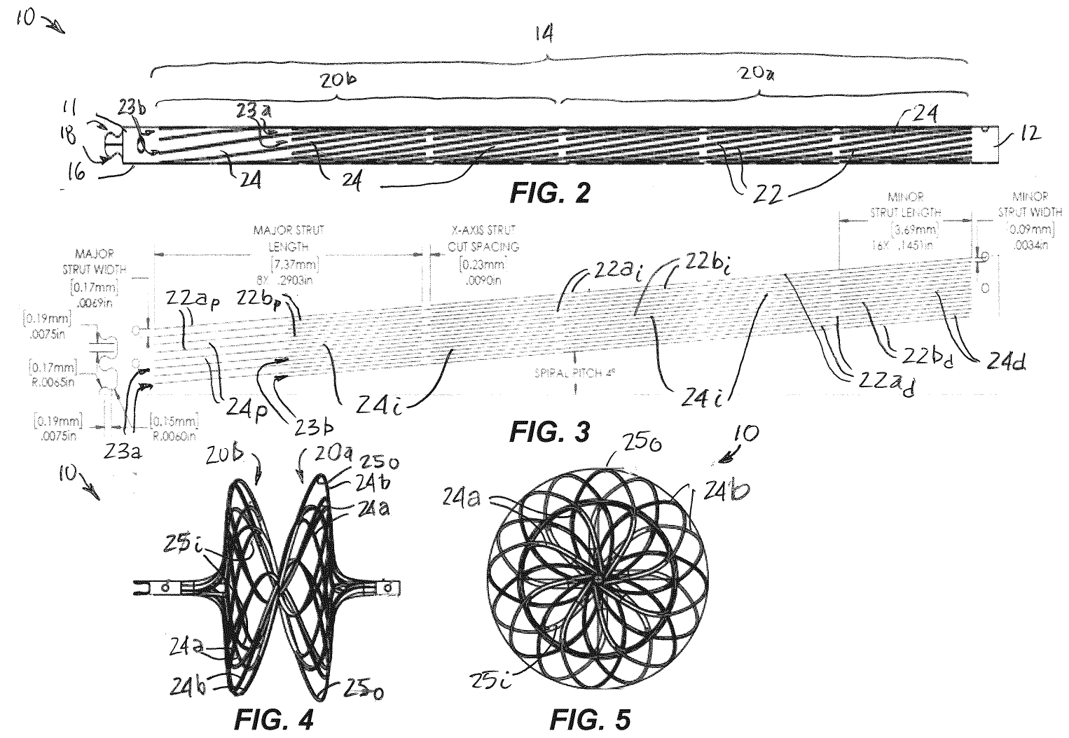

FIG. 2 shows the embolic implant 10 in an unexpanded arrangement, with the proximal end 16 on the left and the distal end 12 on the right. The intermediate section 14 and its adjacent expandable sections 20a and 20b are also illustrated, with the expandable section 20a located adjacent to the distal end 12 and the expandable section 20b located adjacent to the proximal end 16.

The embolic implant 10, including its distal end 12, intermediate section 14, and proximal end 16, may be formed from a tube 11. Without limitation, the tube 11 may comprise a hypotube. In some embodiments, the hypotube may be formed from a metal or a metal alloy. For example, the hypotube may be formed from a shape memory material, such as a shape memory alloy (e.g., a nickel-titanium alloy, nitinol, a nickel-chromium-based superallow (e.g., INCONEL® alloy, etc.) etc.). Such a hypotube may have the unexpanded arrangement at a first temperature (e.g., less than 37° C., below body temperature, at room temperature (e.g., about 25° C.), etc.) and assume the expanded arrangement when heated to a second temperature (e.g., body temperature, 37° C., etc.). As another example, the hypotube may be defined from a stainless steel (e.g., am austenitic stainless steel, such as grade 304 stainless steel, grade 316 stainless steel, grade 316L stainless steel, etc.). In other embodiments, the hypotube may be formed from a suitable polymer (e.g., polyether ether ketone (PEEK), polyimide, polytetrafluoroethylene (PTFE), etc.). Materials such as stainless steel and polymers that are not affected by changes from room temperature to body temperature may comprise a material that, when relaxed, may assume the expanded configuration but may be resiliently constrained into the unexpanded configuration (e.g., by a catheter, etc.).

Each expandable section 20a, 20b of the intermediate section 14 of the embolic implant 10 may be defined by forming a plurality of cuts, or slits 22, through the tube 11. Without limitation, the slits 22 may be formed by laser cutting the tube 11. The slits 22 may be oriented parallel to one another and extend helically around the tube 11. Without limitation, the slits 22 may be arranged at an angle of about 30° or less to a longitudinal axis of the tube 11, at an angle of about 20° or less to the longitudinal axis of the tube 11, at an angle of about 10° or less to the longitudinal axis of the tube 11, or at an angle of about 5° or less (e.g., at 6°, 4°, 3°, 2°, 1°, etc.) to the longitudinal axis of the tube 11. The slits 22 may be oriented in a clockwise helix around the tube 11, in a counterclockwise helix around the tube 11, or in an combination of clockwise and counterclockwise helices around the tube 11.

More specifically, the slits 22 may be oriented parallel to one another and in rows 23a and 23b in which slits 22 are arranged end-to-end. The rows 23a and 23b of slits 22 and the slits 22 of each row 23a, 23b may extend helically around the tube 11. Optionally, as illustrated, the slits 22 and the rows 23a, 23b may extend in counterclockwise helices around the tube 11. The slits 22 of a row 23a, 23b of slits 22 may be offset from the slits 22 of each circumferentially adjacent row 23b, 23a of slits 22. The offsets may be arranged as a so-called “running bond pattern” of slits 22, in which each slit 22 extends along about half of a length of each circumferentially adjacent slit 22. The number of rows 23 of slits 22 around the tube 11 contributes to the stiffness of softness of the embolic implant 10. An increase in the number of rows 23 of slits 22 around the circumference of the tube 11 corresponds to an increase in the softness of the embolic implant 10. A softer embolic implant 10 may pack in unpredictable ways that may create better blockage, or occlusion, of a vessel or a void.

With added reference to FIG. 3, which shows how a pattern of slits 22 in the tube 11 would appear if the tube 11 were cut helically without intersecting any of the slits 22 and then flattened, a specific embodiment of a pattern of slits 22 in the tube 11 is shown and described. The slits 22 may include first slits 22a and second slits 22b. The first slits 22a may define first rows 23a, with each first row 23a including at least one first slit 22a. The second slits 22b may define second rows 23b, with each second row 23b including at least one second slit 22b positioned circumferentially adjacent to a portion of at least one first slit 22a of each circumferentially adjacent first row 23a. The first rows 23a and second rows 23b may alternate with each other around a circumference of the tube 11 to define a plurality of struts 24. Each strut 24 may have a somewhat rectangular shape, which may enable the strut 24 or a portion thereof to occlude a void or passage.

More specifically, in the expandable section 20b of the embolic implant 10, each first row 23a may include a proximal first slit 22aₚ and an intermediate first slit 22aᵢ that are oriented end-to-end, while each second row 23b may include a proximal second slit 22bₚ positioned circumferentially adjacent to a distal portion of the proximal first slit 22aₚ and a proximal portion of the intermediate first slit 22aᵢ of each circumferentially adjacent first row 23a. Each second row 23b may lack a slit circumferentially adjacent to a proximal portion of the proximal first slit 22aₚ of each circumferentially adjacent first row 23a. A proximal portion of each intermediate first slit 22aᵢ may define part of the expandable section 20b, while a distal portion of each intermediate first slit 22aᵢ may define part of the expandable section 20a of the embolic implant 10.

In addition to including the distal portion of each intermediate first slit 22aᵢ, the expandable section 20a of the embolic implant may include a distal first slit 22a_{d} in each first row 23a. The portion of each second row 23b that extends into the expandable section 20a may include an intermediate second slit 22bᵢ and a distal second slit 22b_{d}. A proximal portion of each intermediate second slit 22bᵢ may be positioned adjacent to distal portions of adjacent intermediate first slits 22aᵢ, while a distal portion of each intermediate second slit 23bᵢ may be positioned adjacent to proximal portions of adjacent distal first slits 22a_{d}. Each distal second slit 22b_{d}, which is shorter then every other slit 22, may be positioned adjacent to distal portions of adjacent distal first slits 22a_{d}.

In the specific but nonlimiting embodiment of embolic implant 10 depicted by FIGS. 2 and 3, the slits 22 may be oriented at an angle of 4° to the longitudinal axis of the tube 11; thus, the slits 22 may have a spiral pitch of 4°. Each proximal first slit 22aₚ, intermediate first slit 22aᵢ, distal first slit 22a_{d}, proximal second slit 22bₚ, and intermediate second slit 22bᵢ may have the same length, while the distal second slits 22b_{d} may be shorter than (e.g., about half as long as, etc.) the other slits 22. In a specific embodiment, each proximal first slit 22aₚ, intermediate first slit 22aᵢ, distal first slit 22a_{d}, proximal second slit 22bₚ, and intermediate second slit 22bᵢ may have a length of about 7.4 mm, and each distal second slit 22b_{d} may have a length of about 3.7 mm, with adjacent slits 22 in each row 23 being spaced about 0.2 mm apart from each other. Circumferentially adjacent rows 23 of struts 22 may be positioned about 0.1 mm apart from each other to define intermediate struts 24; and distal struts 24a with widths of about 0.1 mm and proximal struts 24p with widths of about 0.2 mm.

In FIGS. 4 and 5, the expandable sections 20a and 20b of the embolic implant 10 are in expanded arrangements, placing the embolic implant 10 in an expanded arrangement. As shown, the embolic implant 10 is significantly shorter and its outer diameter has increased significantly. In the specific embodiment depicted by FIGS. 2-5, each expandable section 20a, 20b may expand to an outer diameter of about 7 mm to about 8 mm (e.g., 7.36 mm+0.99 mm/−0.41 mm, etc.). Such an embolic implant 10 may extend across and, thus, occlude, a vessel of a void with an inner diameter of at least 5 mm.

In the expanded arrangement, struts 24 of the expandable sections 20a and 20b separate from each other, or are spaced apart from each other, by the slits 22 that define the struts 24. As the struts 24 separate from each other, they move radially, increasing the outer diameter of the expandable section 20a, 20b they are a part of, as well as the outer diameter of the embolic implant 10. In addition, the struts 24 twist, or they rotate about their lengths or longitudinal axes, and fold. With the pattern of slits 22 depicted by FIGS. 2 and 3, first struts 24a fold into inner loops 25i that extend to a first radius 26i and second struts 24b fold into outer loops 250 that extend to a second radius 260 that exceeds the first radius 26i. Such twisting and folding of the struts 24 imparts the expanded embolic implant 10 with the appearance of a flower, or with a flower configuration.

With returned reference to FIG. 1, as an expandable section 20a, 20b expands to its expanded arrangement, the struts 24 may rotate about their longitudinal axes (i.e., twist) and fold into loops 25 in which different locations of a surface of the strut 24 along a length of the strut 24 may face in different directions. In some embodiments, at an apex 26 of each loop 25 (e.g., a midpoint of each loop, etc.), an edge of the strut 24 may face outwardly, placing the apex 26 of the loop 25 in a somewhat radial orientation. With the apex 26 of the loop 25 in the somewhat radial orientation, the edge of the strut 24 may engage an inner surface of a wall (e.g., a wall of a vessel, etc.) against which the apex 26 of the loop 25 is positioned, which may optionally anchor the embolic implant 10 in place at the target location (e.g., within a vessel, etc.). Locations of each strut 24 midway between a base of a loop 25 formed by the strut 24 (e.g., an unexpanded segment of the tube 11, etc.) and an apex 26 of the loop 25 may be oriented transverse to a longitudinal axis of the tube 11. When the longitudinal axis of the tube 11 of the embolic implant 10 is oriented along a direction in which blood flows through the target location with a body of a subject, these surfaces of the strut 24 may be oriented transverse to the direction in which blood flows, which may enable these surfaces of the strut 24 to disrupt the flow of blood through the target location.

FIG. 6 illustrates another embodiment of an embolic implant 10′. The embolic implant 10′ includes a distal end 12′, an intermediate section 14′, and a proximal end 16′. The intermediate section 14′ may be directly adjacent to the distal end 12′. The proximal end 16′ is on an opposite side of the intermediate section 14′ from the distal end 12′. The proximal end 16′ may be directly adjacent to the intermediate section 14′.

The intermediate section 14′ of the embolic implant 10′ includes at least one expandable section 20′. In the embodiment of embolic implant 10′ illustrated by FIG. 1, the intermediate section 14′ includes three expandable sections 20a′, 20b′, and 20c′ that, as illustrated, are expanded and, thus, in expanded arrangements. The intermediate section 14′ also includes a non-expandable section 21′, which may be located between expandable sections 20b′ and 20c′.

The proximal end 16′ of the embolic implant 10′ may include a release 18′, which enables the embolic implant 10′ to be coupled to a delivery device (not shown) that delivers the embolic implant to a target location (e.g., a location within a body of a subject, etc.). The release 18′ may have a configuration that enables the embolic implant 10′ to be released by the delivery device once the embolic implant 10′ has been advanced to and, optionally, positioned within the target location. In some embodiments, a configuration of the release 18′ may also enable the delivery device to reengage the embolic implant 10′ to facilitate its repositioning within the target location, movement within the target location or to another location, or removal (e.g., from the body of the subject, etc.).

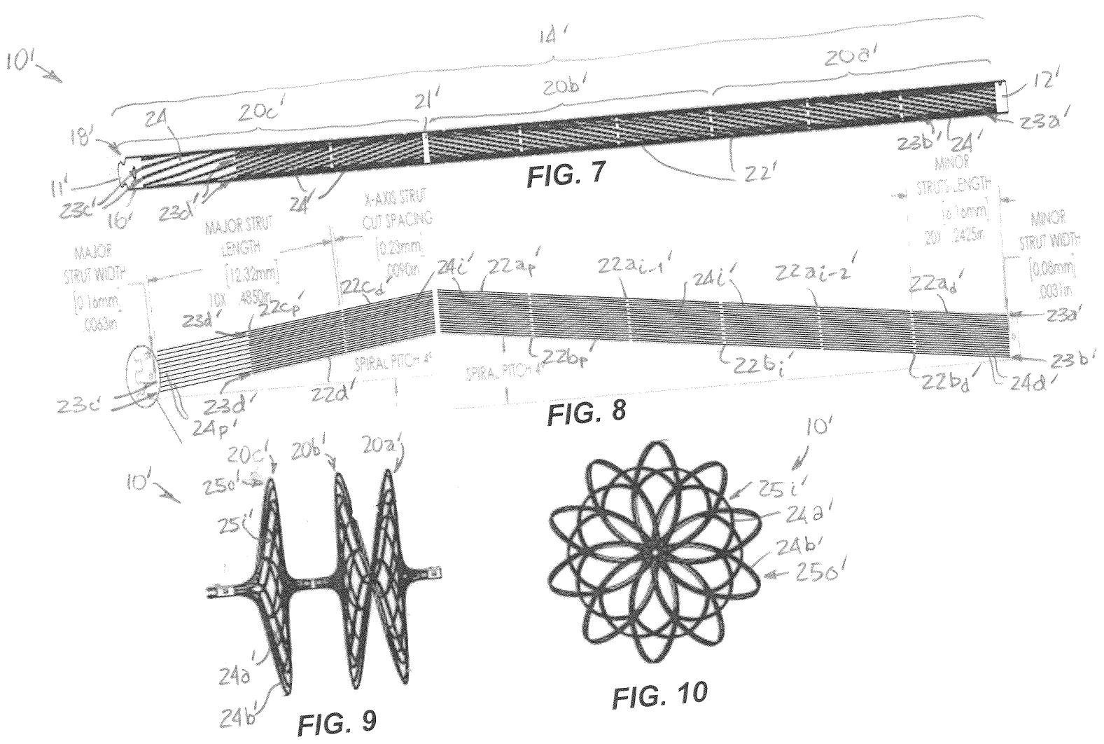

FIG. 7 shows the embolic implant 10′ in an unexpanded arrangement, with the proximal end 16′ on the left and the distal end 12′ on the right. The intermediate section 14′, its expandable sections 20a′, 20b′, and 20c′, and its non-expandable section 21′ are also illustrated, with the expandable section 20a′ located adjacent to the distal end 12, the expandable section 20b′ located adjacent to the expandable section 20a′, the non-expandable section 21′ located adjacent to the expandable section 20b′, and the expandable section 20c′ located adjacent to the non-expandable section 21′ and the proximal end 16′.

The embolic implant 10′, including its distal end 12′, intermediate section 14′, and proximal end 16′, may be formed from a tube 11′. Without limitation, the tube 11′ may comprise a hypotube. In some embodiments, the hypotube may be formed from a metal or a metal alloy. For example, the hypotube may be formed from a shape memory material, such as a shape memory alloy (e.g., a nickel-titanium alloy, or nitinol, etc.). As another example, the hypotube may be defined from a stainless steel (e.g., am austenitic stainless steel, such as grade 304 stainless steel, grade 316 stainless steel, grade 316L stainless steel, etc.). In other embodiments, the hypotube may be formed from a suitable polymer (e.g., polyether ether ketone (PEEK), polyimide, polytetrafluoroethylene (PTFE), etc.).

Each expandable section 20a′, 20b′, 20c′ of the intermediate section 14′ of the embolic implant 10′ may be defined by forming a plurality of cuts, or slits 22′, through the tube 11′. Without limitation, the slits 22′ may be formed by laser cutting the tube 11′. The slits 22′ may be oriented parallel to one another and extend helically around the tube 11′. Without limitation, the slits 22′ may be arranged at an angle of about 30° or less to a longitudinal axis of the tube 11′, at an angle of about 20° or less to the longitudinal axis of the tube 11′, at an angle of about 10° or less to the longitudinal axis of the tube 11′, or at an angle of about 5° or less (e.g., at 6°, 4°, 3°, 2°, 1°, etc.) to the longitudinal axis of the tube 11′. The slits 22′ may be oriented in a clockwise helix around the tube 11′, in a counterclockwise helix around the tube 11′, or in an combination of clockwise and counterclockwise helices around the tube 11′.

More specifically, the slits 22′ may be oriented parallel to one another and in rows 23a′, 23b′, 23c′, and 23d′, in which slits 22′ are arranged end-to-end. The slits 22′ and the rows 23a′, 23b′, 23c′, and 23d′ they define may extend helically around the tube 11′. More specifically, the rows 23a′ and 23b′ may be oriented as clockwise helices around the tube 11′, while the rows 23c′ and 23d′ may be oriented as counterclockwise helices around the tube 11′. The slits 22′ of a row 23a′, 23b′; 23c′, 23d′ of slits 22′ may be offset from the slits 22′ of each circumferentially adjacent row 23b′, 23a′; 23d′, 23c′. The offsets may be arranged as so-called “running bond patterns” of slits 22′, in which each slit 22′ extends along about half of a length of each circumferentially adjacent slit 22′. The number of rows 23′ of slits 22′ around the tube 11′ contributes to the stiffness of softness of the embolic implant 10′. An increase in the number of rows 23′ of slits 22′ around the circumference of the tube 11′ corresponds to an increase in the softness of the embolic implant 10′. A softer embolic implant 10′ may pack in unpredictable ways that may create better blockage, or occlusion, of a vessel or a void.

With added reference to FIG. 8, which shows how a pattern of slits 22′ in the tube 11′ would appear if the tube 11′ were cut helically without intersecting any of the slits 22′ and then flattened, a specific embodiment of a pattern of slits 22′ in the tube 11′ is shown and described. The slits 22′ that define the expandable sections 20a′ and 20b′ may include first slits 22a′ and second slits 22b′. The first slits 22a′ may define first rows 23a′, with each first row 23a′ including at least one first slit 22a′. The second slits 22b′ may define second rows 23b′, with each second row 23b′ including at least one second slit 22b′ positioned circumferentially adjacent to a portion of at least one first slit 22a′ of each circumferentially adjacent first row 23a′. The first rows 23a′ and second rows 23b′ may alternate with each other around a circumference of the tube 11′ to define a plurality of struts 24′. Each strut 24′ may have a somewhat rectangular shape, which may enable the strut 24′ or a portion thereof to occlude a void or passage.

The slits 22′ that define the expandable section 20c′ may include third slits 22c′ and fourth slits 22d′. The third slits 22c′ may define third rows 23c′, with each third row 23c′ including at least one third slit 22c′. The fourth slits 22d′ may define fourth rows 23d′, with each fourth row 23d′ including at least one fourth slit 22d′ positioned circumferentially adjacent to a portion of at least one third slit 22c′ of each circumferentially adjacent third row 23c′. The third rows 23c′ and fourth rows 23d′ may alternate with each other around a circumference of the tube 11′ to define a plurality of struts 24′.

More specifically, in the expandable section 20c′ of the intermediate section 14′ of the embolic implant 10′, each third row 23c′ may include a proximal third slit 22cₚ′ and a distal third slit 22c_{d}′ that are oriented end-to-end, while each fourth row 23d′ may include a fourth slit 22d′ positioned circumferentially adjacent to a distal portion of the proximal third slit 22cₚ′ and the distal third slit 22c_{d}′ of each circumferentially adjacent third row 23c′. Each fourth row 23d′ may lack a slit circumferentially adjacent to a proximal portion of the proximal third slit 22cₚ′ of each circumferentially adjacent first row 23c′. Distal ends of the distal third slits 22c_{d}′ and fourth slits 22d′ may be located adjacent to the non-expandable section 21′ of the intermediate section 14′.

On the opposite side of the non-expandable section 21′ of the intermediate section 14′ of the tube 11′ from which the embolic implant 10′ is defined, each first row 23a′ may include a proximal first slit 22aₚ′, two intermediate first slits 22aᵢ₋₁′ and 22aᵢ₋₂′, and a distal first slit 22a_{d}′ that are oriented end-to-end. Each second row 23b′ may include a proximal second slit 22bₚ′, an intermediate second slit 22bᵢ′, and a distal second slit 22b_{d}′ that are positioned end-to-end. Each proximal second slit 22bₚ′ may be positioned circumferentially adjacent to the proximal first slit 22aₚ′ and a proximal portion of an intermediate first slit 22aᵢ₋₁′ of each circumferentially adjacent first row 23a′. A proximal portion of each intermediate second slit 22bᵢ′ may be positioned circumferentially adjacent to a distal portion of the intermediate first slit 22aᵢ₋₁′ of each circumferentially adjacent first row 23a′, while a distal portion of each intermediate second slit 22bᵢ′ may be positioned circumferentially adjacent to a proximal portion of another intermediate first slit 22aᵢ₋₂′ of each circumferentially adjacent first row 23a′. A proximal portion of each distal second slit 22b_{d}′ may be positioned circumferentially adjacent to a distal portion of the other intermediate first slit 22aᵢ₋₂′ of each circumferentially adjacent first row 23a′, while a distal portion of each distal second slit 22b_{d}′ may be positioned circumferentially adjacent to the distal first slit 22a_{d}′ of each circumferentially adjacent first row 23a′.

The proximal first slit 22aₚ′ and intermediate first slit 22aᵢ₋₁′ of each first row 22a′ and the proximal second slit 22bₚ′ and a proximal portion of the intermediate second slit 22bᵢ′ of each second row 23b′ may define the expandable section 20b′ of the intermediate section 14′ of the embolic implant 10′. The other intermediate first slit 22aᵢ₋₂′ and distal first slit 22a_{d}′ of each first row 22a′ and the distal portion of the intermediate second slit 22bᵢ′ and the distal second slit 22dᵢ′ of each second row 23a′ may define the expandable section 22a′ of the intermediate section 14′ of the embolic implant 10′.

In the specific but nonlimiting embodiment of embolic implant 10′ depicted by FIGS. 7 and 8, the slits 22′ may be oriented at an angle of 4° to the longitudinal axis of the tube 11′; thus, the slits 22′ may have a spiral pitch of 4°. Each proximal third slit 22cₚ′, proximal fourth slit 22dₚ′, intermediate first slit 22aᵢ₋₁′, other intermediate first slit 22aᵢ₋₂′, proximal second slit 22bₚ′, intermediate second slit 22bᵢ′, and proximal second slit 22bₚ′ may have the same length, while the distal third slits 22c_{d}′, proximal first slits 22aₚ′, and distal first slits 22a_{d}′ may be shorter than (e.g., about half as long as, etc.) the other slits 22′. In a specific embodiment, each proximal third slit 22cₚ′, proximal fourth slit 22dₚ′, intermediate first slit 22aᵢ₋₁′, other intermediate first slit 22aᵢ₋₂′, proximal second slit 22bₚ′, intermediate second slit 22bᵢ, and proximal second slit 22bₚ may have a length of about 12.3 mm, and each distal third slit 22c_{d}′, proximal first slit 22aₚ′, and distal first slit 22a_{d}′ may have a length of about 6.2 mm, with adjacent slits 22′ in each row 23′ being spaced about 0.2 mm apart from each other. Circumferentially adjacent rows 23′ of struts 22′ may be positioned about 0.1 mm apart from each other to define intermediate struts 24i′ and distal struts 24d′ with widths of about 0.1 mm and proximal struts 24p′ with widths of about 0.2 mm.

In FIGS. 9 and 10, the expandable sections 20a′, 20b′, and 20c′ of the embolic implant 10′ are in expanded arrangements, placing the embolic implant 10′ in an expanded arrangement. As shown, the embolic implant 10′ is significantly shorter and its outer diameter has increased significantly. In the specific embodiment depicted by FIGS. 7-10, each expandable section 20a′, 20b′, 20c′ may expand to an outer diameter of about 12 mm to about 13 mm (e.g., 12.35 mm±0.51, etc.). Such an embolic implant 10 may extend across and, thus, occlude, a vessel of a void with an inner diameter of at least 8 mm.

In the expanded arrangement, struts 24′ of the expandable sections 20a′, 20b′, and 20c′ separate from each other, or are spaced apart from each other, by the slits 22′ that define the struts 24′. As the struts 24′ separate from each other, they move radially, increasing the outer diameter of the expandable section 20a′, 20b′, 20c′ they are a part of, as well as the outer diameter of the embolic implant 10′. In addition, the struts 24′ twist, or they rotate about their lengths or longitudinal axes, and fold. With the pattern of slits 22′ depicted by FIGS. 7 and 8, first struts 24a′ fold into inner loops 25i′ that extend to a first radius 26i′ and second struts 24b′ fold into outer loops 250′ that extend to a second radius 260′ that exceeds the first radius 26i′. Such twisting and folding of the struts 24′ imparts the expanded embolic implant 10′ with the appearance of a flower, or with a flower configuration.

With returned reference to FIG. 6, as an expandable section 20a′, 20b′ expands to its expanded arrangement, the struts 24′ may rotate about their longitudinal axes (i.e., twist) and fold into loops 25′ in which different locations of a surface of the strut 24′ along a length of the strut 24′ may face in different directions. In some embodiments, at an apex 26′ of each loop 25′ (e.g., a midpoint of each loop, etc.), an edge of the strut 24′ may face outwardly, placing the apex 26′ of the loop 25′ in a somewhat radial orientation. With the apex 26′ of the loop 25′ in the somewhat radial orientation, the edge of the strut 24′ may engage an inner surface of a wall (e.g., a wall of a vessel, etc.) against which the apex 26′ of the loop 25′ is positioned, which may optionally anchor the embolic implant 10′ in place at the target location (e.g., within a vessel, etc.). Locations of each strut 24′ midway between a base of a loop 25′ formed by the strut 24′ (e.g., an unexpanded segment of the tube 11′, etc.) and an apex 26′ of the loop 25′ may be oriented transverse to a longitudinal axis of the tube 11′. When the longitudinal axis of the tube 11′ of the embolic implant 10′ is oriented along a direction in which blood flows through the target location with a body of a subject, these surfaces of the strut 24′ may be oriented transverse to the direction in which blood flows, which may enable these surfaces of the strut 24′ to disrupt the flow of blood through the target location.

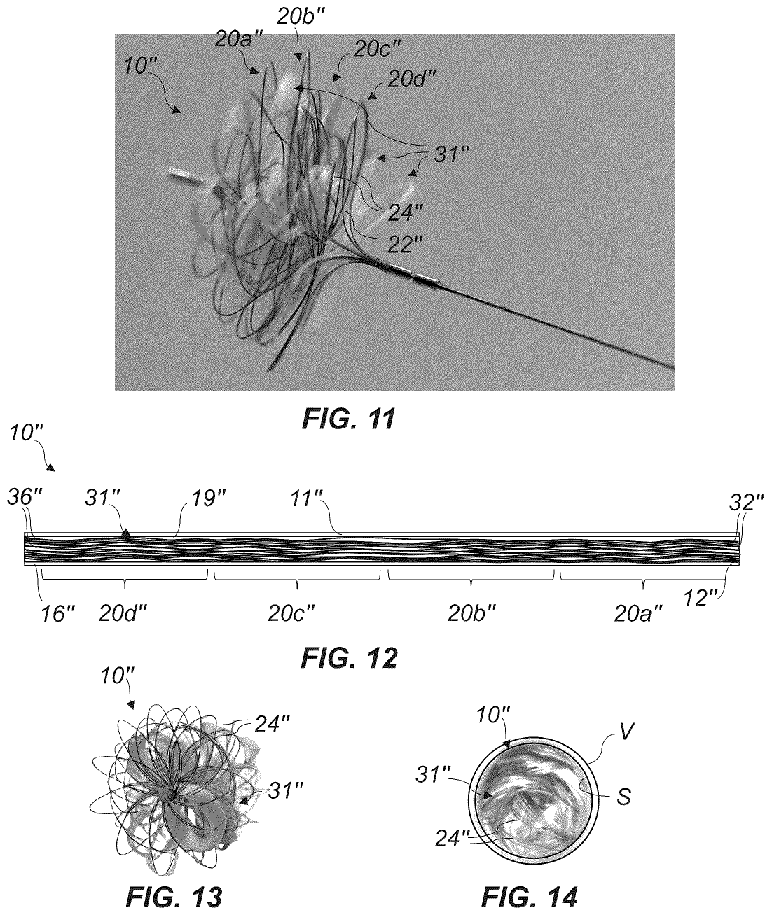

Turning now to FIGS. 11-14, an embodiment of an embolic implant 10″ that includes a filler 30″ throughout its length is depicted. The embolic implant 10″ includes four expandable sections 20a″, 20b″, 20c″, and 20d″, each defined from a section of a tube 11″ (FIGS. 12 and 13) that has been cut to define slits 22″ and struts 24″. The filler 30″ includes fibers 31″. FIG. 11 shows the embolic implant 10″ in its expanded arrangement. FIG. 12 shows the embolic implant 10″ in its unexpanded arrangement.

As shown in FIG. 12, the fibers 31″ may substantially fill a lumen 19″ of the tube 11″. Each fiber 31″ may extend along an entire length of the tube 11″. A distal end 32″ of the fibers 31″ may be constrained within a distal end 12″ of the tube 11″ and embolic implant 10″ (e.g., mechanically, with glue, etc.). A proximal end 36″ of the fibers 31″ may be constrained within a proximal end 16″ of the tube 11″ and embolic implant 10″ (e.g., mechanically, with glue, etc.). The fibers 31″ may comprise individual fibers 31″ or they may be continuously looped or repeatedly folded, with a single fiber 31″ passing back and forth through the interior of the embolic implant 10″. Without limitation, a repeated folded fiber 31″ may comprise a long fiber that has been repeatedly folded in half. A fiber 31″ that has been folded in half sixteen times may provide 324 fiber lengths along the length of the embolic implant 10″. A fiber 31″ that has been folded in half twenty-two times may provide 484 fiber lengths along the length of the embolic implant. Thus, about 300 fibers 31″ or fiber 31″ lengths to about 500 fibers 31″ or fiber 31″ lengths may span the entire length of the embolic implant 10″.

The fibers 31″ may be manipulated in one or more ways to enhance the extent to which the fibers 31″ spread out and interact with each other and with the struts 24″ as the embolic implant 10″ expands. In such embodiments, the fibers 31″ may be longer than the tube 11″. For example, the fibers 31″ may be collectively twisted to define one or more helices within the lumen 19″. As another example, the fibers 31″ may be packed into the lumen 19″ in a manner that causes them to collapse lengthwise within the lumen 19″. Other techniques that enhance the extent to which the fibers 31″ spread out and interact with each other and with the struts 34″ as the embolic implant 10″ expands are also within the scope of this disclosure.

In a specific embodiment, the fibers 31″ may comprise polyester fibers (e.g., polyethylene terephthalate (PET) fibers, etc.). The fibers 31″ may be wavy fibers, which may include kinks that enhance their ability to fill the interior of the embolic implant 10″ as it expands, as shown in FIGS. 11, 13, and 14. FIGS. 11 and 13 show the embolic implant 10″ in its expanded arrangement without any confinement, while FIG. 14 shows the embolic implant 10″ in its expanded arrangement within a confined space, such as the confines defined at least partially by the surfaces S of a blood vessel V.

As each expandable section 20a″, 20b″, 20c″, 20d″ of the embolic implant 10″ expands, the struts 24″ of the embolic implant 10″ twist, fold, and interact with each other in a manner that forms a framework (e.g., a somewhat spiral frame, etc.) for the fibers 31″ as the distal ends 32″ and proximal ends 36″ of the fibers 31″ are pushed together and the fibers 31″ are spread apart and bunched together. The combination of the framework defined by the struts 24″ and the bunched fibers 31″ supported by the framework may enhanced embolization caused by the embolic implant 10″.

With returned reference to FIGS. 1 and 2, a method of manufacturing an embolic implant 10 may include cutting a plurality of rows 23 of slits 22 into a tube 11 (e.g., a hypotube formed from a shape memory alloy, a hypotube formed from stainless steel, a tube formed from a polymer, etc.) to define at least one expandable section 20a, 20b, etc., from the tube 11. Optionally, cutting may comprise laser cutting the tube 11. Each slit 22 of the plurality of rows 23 may extend substantially along a longitudinal axis of the tube 11. Optionally, the plurality of rows 23 may extend helically around the tube 11 (e.g., at an angle of about 30° or less to a longitudinal axis of the tube, at an angle of about 20° or less to the longitudinal axis of the tube, at an angle of about 10° or less to the longitudinal axis of the tube, at an angle of about 5° or les to the longitudinal axis of the tube 11, etc.) (e.g., the slits 22 may be oriented in a clockwise helix around the tube 11, etc.). The slits 22 may be cut to define alternating first rows 23a and second rows 23b in the tube 11. Each first row 23a may include a series of first slits 22a arranged end-to-end. Each second row 23b may include a series of second slits 22b arranged end-to-end. Each second slit 22b may be longitudinally offset from circumferentially adjacent first slits 22a of each adjacent first row 23a. The first rows 23a and second rows 23b define struts 24 of the expandable section(s) 20a, 20b, etc., with an arrangement of the first rows 23a and the second rows 23b enabling the expandable section 20a, 20b, etc., to expand to an expanded arrangement. Without limitation, as shown in FIGS. 4 and 5, the expanded arrangement may comprise a flower configuration, in which first struts 24a of the expandable section 20a, 20b, etc., fold into inner loops 25i that extend to a first radius and second struts 24b of the expandable section 20a, 20b, etc., fold into outer loops 250 that extend to a second radius that exceeds the first radius.

The method may also include expanding the expandable section 20a, 20b, etc., to its expanded arrangement and setting a shape of the expanded arrangement. In embodiments where the tube 11 comprises a shape memory alloy, the shape may be set thermally, which may enable the shape memory alloy to assume its expanded arrangement when the shape memory alloy is heated to a shape memory temperature (e.g., body temperature, etc.) and the expandable section 20a, 20b, etc., is removed from any physical constraint (e.g., a delivery device, etc.). In some embodiments where the tube 11 comprises another material (e.g. stainless steel, a polymer, etc.), the shape of the expanded arrangement may be set mechanically and/or with heat to make the expanded arrangement the relaxed state of the expandable section 20a, 20b, etc.; the expandable section 20a, 20b, etc., may then be constrained (e.g., in another tube, etc.) until it is delivered to a target site within a body of a subject. In other embodiments where the tube 11 comprises the other material (e.g., stainless steel, a polymer, etc.), the expandable section 20a, 20b, etc., may be substantially tubular in its relaxed state, then mechanically forced into and secured in its expanded arrangement once the embolic implant 10 is delivered to the target site within the subject's body. In each embodiment, placement of the expandable section 20a, 20b, etc., into its expanded arrangement may include forcing opposite ends of the expandable section 20a, 20b, etc., and, optionally, rotating one end of the expandable section about the longitudinal axis of the tube 11 and relative to the other end of the expandable section 20a, 20b, etc., or rotating one end of the tube 11 about the longitudinal axis relative to the other end of the tube 11. The end of the tube 11 may be rotated in the same direction as the helices that are defined by the slits 22 and struts 24 formed from the tube 11 rotate. Such rotation may be up to about 30°.

In embodiments where the embolic implant 10″ includes fibers 31″, such as the embodiment illustrated by FIGS. 11-14, the method of manufacturing the embolic implant 10″ includes introducing the fibers 31″ into the lumen 19″ of the tube 11″ from which the embolic implant 10″ is defined and constraining (e.g., mechanically securing, gluing, etc.) distal ends 32″ of the fibers 31″ within a distal end 12″ of the tube 11″ and embolic implant 10″ and proximal ends 36″ of the fibers 31″ within a proximal end 16″ or the tube 11″ and embolic implant 10″. Introduction of the fibers 31″ may comprise grouping individual fibers and introducing them into the lumen 19″ or repeatedly folding or continuously looping one or more longer fibers and introducing them into the lumen 19″.

Use of an embolic implant 10, 10′, 10″ of this disclosure includes introducing the embolic implant 10, 10′, 10″ to a target location or adjacent to (e.g., proximally adjacent to, etc.) the target location. The target location may be a target location within a body of a subject (e.g., a blood vessel, another vessel or tube, a void, etc.). The embolic implant 10, 10′, 10″ may be secured to a distal end of a delivery device (not shown) (e.g., a control wire, etc.) that can selectively release the embolic implant 10, 10′, 10″.

A distal end of a catheter or another suitable vascular access device (e.g., a sheath, a cannula, a needle, etc.) may be advanced to target location or to a location adjacent to (e.g., proximally adjacent to, etc.) a target location within a body of a subject.

The delivery device and embolic implant 10, 10′, 10″ may be introduced through the catheter (not shown) (e.g., a catheter with an inner diameter (ID) of about 0.027 in or about 0.7 mm, etc.) or other vascular access device. Once a distal tip of the catheter or other suitable vascular access device is present at the target location or adjacent to the target location, the embolic implant 10, 10′, 10″ may be advanced distally out of the distal tip of the catheter or other vascular access device. The embolic implant 10, 10′, 10″ may automatically expand (e.g., upon releasing a constraining force placed on the embolic implant 10, 10′, 10″ by the catheter or other vascular access device, upon exposing the embolic implant 10, 10′, 10″ to an expanding condition present at the target location (e.g., body temperature, etc.), etc.) once the embolic implant 10, 10′, 10″ exits the catheter or it may be mechanically expanded with the delivery device (e.g., by forcing the proximal end 16, 16′, 16″ of the embolic implant 10, 10′, 10″ toward the distal end 12, 12′, 12″ of the embolic implant 10, 10′, 10″, etc.). More specifically, an outer diameter of the tube 11, 11′, 11″ of the embolic implant 10, 10′, 10″ expands and a length of the embolic implant 10, 10′, 10″ contracts, placing the embolic implant 10, 10′, 10″ in its expanded arrangement. As the embolic implant 10, 10′, 10″ expands, it may assume a final shape, which is at least partially defined by the slits 22, 22′, 22″ in the tube 11, 11′, 11″ and the boundaries of the space in which the embolic device 10, 10′, 10″ expands. In some embodiments, including those where the embolic device 10, 10′, 10″ includes a plurality of closely positioned expandable sections 20a, 20a′, 20a″; 20b, 20b′, 20b″; etc., the expandable sections 20a, 20a′, 20a″; 20b, 20b′, 20b″; etc., which may define mesh disks, may pack upon each other, or mesh together; thus, the embolic device 10, 10′, 10″ may pack upon itself in its final shape.

Optionally, the embolic implant 10, 10′, 10″ may be repositioned or reoriented. In some embodiments, the embolic implant 10, 10′, 10″ may be contracted, moved, repositioned, and/or reexpanded.

Once the embolic implant 10, 10′, 10″ is in place at the target location, the embolic implant 10, 10′, 10″ may be released from the delivery device. The delivery device may then be removed from the body of the subject. As an example, the embolic implant 10, 10′, 10″ may be disconnected from the delivery device by pulling the delivery device proximally to pull the embolic implant 10, 10′, 10″ against the proximal end of the catheter or other vascular access device with sufficient force to allow the embolic implant 10, 10′, 10″ to break away from or otherwise uncouple from a distal portion of the delivery device. As another example, the delivery device may be manipulated (e.g., rotated, a button pushed, etc.) to mechanically release the embolic implant 10, 10′, 10″. As yet another example, expansion of the embolic implant 10, 10′, 10″ may at least partially facilitate disconnection of the embolic implant 10, 10′, 10″ from the distal portion of the delivery device. Once the distal portion of the delivery device disengages the embolic implant 10, 10′, 10″ (e.g., a release on a proximal end of the embolic implant 10, 10′, 10″, etc.), the delivery device may be withdrawn proximally through the catheter or other vascular access device.

Optionally, one or more additional embolic implants 10, 10′, 10″ may be delivered to the target location in the same way. Once delivery of embolic implants 10, 10′, 10″ to the target location is complete, the catheter may be removed from the body of the subject.

Although the disclosure provides many specifics, the specifics should not be construed as limiting the scope of any of the claims, but merely as providing illustrations of some embodiments of elements and features of the disclosed subject matter that fall within the scopes of the claims. Other embodiments of the disclosed subject matter may be devised that are also within the scopes of the claims. Accordingly, the scope of each claim is limited only by its plain language and the legal equivalents thereto.

What is claimed:

1. An embolic implant, comprising:
a hypotube including a wall and having a length with distal end and a proximal end, the wall defining a lumen through the length of the hypotube; and
at least one expandable section along the length of the hypotube defined by a plurality of slits cut into the wall, extending along the length of the hypotube, oriented parallel to each other and helically around the hypotube, with circumferentially adjacent rows of slits being offset from each other, the plurality of slits defining struts that fold to cause a first location of a surface of a strut of the struts to substantially face a second location of the surface of the strut.

2. The embolic implant of claim 1, wherein the plurality of slits is oriented at an angle of about 5° or less to the longitudinal axis of the hypotube.

3. The embolic implant of claim 1, wherein the plurality of slits is oriented in a clockwise helix around the hypotube.

4. The embolic implant of claim 1, wherein the struts defined by the plurality of slits fold upon expansion of the at least one expandable section.

5. The embolic implant of claim 4, wherein a center of an edge of each strut of the struts rotates outwardly to a circumferential position upon expansion of the at least one expandable section.

6. The embolic implant of claim 1, wherein the plurality of slits defines alternating first rows of slits and second rows of slits around a circumference of the hypotube.

7. The embolic implant of claim 6, wherein first slits arranged end-to-end in a first row of slits of the first rows of slits are offset from second slits arranged end-to-to end in a second row of slits of the second rows slits, the second row of slits being positioned adjacent to the first row of slits.

8. The embolic implant of claim 7, wherein at least some of the first slits extend about halfway along at least some adjacent second slits of the second slits.

9. The embolic implant of claim 8, wherein the plurality of slits includes:
major slits of substantially a first length; and
minor slits of substantially a second length equal to about half the first length, the minor slits being located at a distal end and/or a proximal end of the at least one expandable section and comprising half of all slits extending to the distal end and/or the proximal end of the at least one expandable section.

10. The embolic implant of claim 9, wherein minor slits of the first rows of slits extend about halfway along circumferentially adjacent major slits of the second rows of slits.

11. The embolic implant of claim 10, wherein the at least one expandable section has an expanded arrangement in which the struts assume a flower configuration.

12. The embolic implant of claim 11, wherein the flower configuration includes first struts of the struts of the at least one expandable section that fold into inner loops that extend to a first radius and second struts of the struts of the at least one expandable section that fold into outer loops that extend to a second radius that exceeds the first radius.

13. The embolic implant of claim 1, comprising a plurality of expandable sections along the length of the hypotube.

14. The embolic implant of claim 1, wherein the at least one expandable section rotates about a longitudinal axis of the hypotube upon expanding.

15. The embolic implant of claim 1, further comprising:
fibers extending along an entire length of the hypotube and constrained by the distal end and the proximal end of the hypotube.

16. The embolic implant of claim 15, wherein the fibers comprise a continuously looped or repeatedly folded fiber.

17. The embolic implant of claim 15, wherein the fibers comprise wavy fibers.

18. An embolic implant, comprising:
a hypotube including a wall and having a length with distal end and a proximal end, the wall defining a lumen through a length of the hypotube; and
an expandable section along the length of the hypotube defined by a plurality of slits cut into the wall, extending along the length of the hypotube, and oriented parallel to each other and helically around the hypotube, the plurality of slits including:
first slits defining first rows of slits, each first row of the first rows of slits including at least one first slit; and
second slits defining second rows of slits, each second row of the second rows of slits including at least one second slit positioned circumferentially adjacent to a portion of the at least one first slit of each circumferentially adjacent first row,
the first rows of slits and second rows of slits alternating with each other around a circumference of the hypotube to define a plurality of struts, each strut of the plurality of struts being foldable upon expansion of the expandable section.

19. The embolic implant of claim 18, wherein the expandable section has an expanded arrangement in which includes first struts of the plurality of struts of the at least one expandable section that fold into inner loops that extend to a first radius and second struts of the plurality of struts of the at least one expandable section that fold into outer loops that extend to a second radius that exceeds the first radius.

20. The embolic implant of claim 19, further comprising:
another expandable section defined by another plurality of slits cut into the wall of the hypotube, the another plurality of slits including:
first slits defining first rows of slits, each first row of the first rows of slits including at least one first slit; and
second slits defining second rows of slits, each second row of the second rows of slits including at least one second slit positioned circumferentially adjacent to a portion of the at least one first slit of each circumferentially adjacent first row,
the first rows of slits and second rows of slits alternating with each other around a circumference of the hypotube to define another plurality of struts that extend along the length of the length of the hypotube, the another plurality of struts foldable upon expansion of the another expandable section.

21. A method for manufacturing an embolic implant, comprising:
cutting a plurality of rows of slits into a tube to define an expandable section from the tube, each slit of the plurality of rows of slits extending substantially along a longitudinal axis of the tube, including cutting alternating first rows of slits and second rows of slits,
each first row of slits of the first rows of slits including a series of first slits arranged end-to-end;
each second row of slits of the second rows of slits including a series of second slits arranged end-to-end, each second slit longitudinally offset from circumferentially adjacent first slits of each adjacent first row of slits of the first rows of slits;
the first rows of slits and second rows of slits defining struts of the expandable section;
an arrangement of the first rows of slits and the second rows of slits enabling the expandable section to expand to an expanded arrangement.

22. The method of claim 21, wherein cutting the plurality of rows of slits comprises cutting the plurality of rows of slits to extend helically around the tube.

23. The method of claim 22, wherein cutting the plurality of rows of slits to extend helically around the tube comprises cutting the plurality of rows of slits to be offset at an angle of about 5° or less from the longitudinal axis of the tube.

24. The method of claim 21, further comprising:
expanding the expandable section to the expanded arrangement; and
setting a shape of the expandable section in the expanded arrangement.

25. The method of claim 24, wherein expanding the expandable section into the expanded arrangement comprises expanding the expandable section into a flower configuration with first struts of the struts of the expandable section folding into inner loops that extend to a first radius and second struts of the struts of the expandable section folding into outer loops that extend to a second radius that exceeds the first radius.

26. The method of claim 25, wherein expanding the expandable section comprises forcing ends of the expandable section toward each other.

27. The method of claim 26, wherein expanding the expandable section further comprises rotating at least one end of the expandable section about a longitudinal axis of the tube and relative to another end of the expandable section.

28. The method of claim 21, further comprising:
introducing fibers into a lumen of the hypotube, each fiber of the fibers extending along substantially an entire length of the hypotube;
constraining distal ends of the fibers in a distal end of the hypotube; and
constraining proximal ends of the fibers in a proximal end of the hypotube.
